(19) United States
(12) Patent Application Publication
(10) Pub. No.: US 2010/0331613 A1
(43) Pub. Date: Dec. 30, 2010

(54) MEDICAL IMPLANT

(75) Inventors: Carlo Ricchardo Centonze, Zurich (CH); Murray Height, Zurich (CH); Ingo Berndt, Tuttlingen (DE); Erich Odermatt, Schaffhausen (CH)

Correspondence Address: IP GROUP OF DLA PIPER LLP (US), ONE LIBERTY PLACE, 1650 MARKET ST, SUITE 4900, PHILADELPHIA, PA, 19103, US

(73) Assignees: AESCULAP AG, Tuttlingen/Donau (DE); HEIQ MATERIALS AG, Bad Zurzach (CH)

(21) Appl. No.: 12/740,417

(22) PCT Filed: Oct. 29, 2008
(86) PCT No.: PCT/EP2008/009111

§ 371 Date: Sep. 3, 2010

(30) Foreign Application Priority Data
Oct. 29, 2007 (DE) 10 2007 052 519.4

(51) Int. Cl. A61F 13/00; A61F 2/02; A61F 2/82

(52) U.S. Cl. 600/37; 623/11.11; 623/1.46

(57) ABSTRACT

The present invention relates to a medical implant which is equipped with an antimicrobial composition which comprises silicon dioxide and metal-containing nanoparticles, and processes for producing the medical implant.

The present invention relates to a medical implant which is equipped with an antimicrobial composition, corresponding production processes and the use of the antimicrobial composition for producing a medical implant.

Post-operative primary infections and also in particular secondary infections are one of the greatest complications in modern maintenance surgery. Hence there has recently been an increasing move towards equipping surgical implants, in particular suture materials and nets, with suitable antimicrobial active substances. Antimicrobial implants of this type are for example described in DE 10 2004 047 568 A1.

Among the post-operative infections, the main problem is posed by secondary infections, which can often only arise after several weeks or even months. This is often a major clinical problem above all with non-absorbable implants which are designed for prolonged residence in the body. Hence in some cases these implants have to be explanted again in order to be able successfully to treat the infections that have arisen. Firstly, this leads to higher treatment costs. Secondly, every further surgical intervention means additional stress and above all a longer hospital stay for the patients concerned. Hence the antimicrobial equipping of implants, in particular of non-absorbable implants, is a rational and necessary further development in the field of implant technology.

However, a problem is that, depending on the antimicrobial active substances, an accumulated, i.e. short-term and (excessively) high dosage release of the active substances into the surrounding biological tissue can take place. This can cause tissue changes ranging from inflammatory to necrotic. In addition, the integration of an implant in the body can be adversely affected by an accumulated active substance release.

A further disadvantage mainly relates to textile implants, for example suture materials, vascular prostheses and nets and bands for hernia, prolapse and incontinence therapy. There, potential weak points in the implant can arise during the incorporation of metal particles which exceed a certain particle size, as a result of which its mechanical strength is decreased. This applies above all for fine suture materials with relatively thin fiber diameters.

Hence the purpose of the present invention is to provide an antimicrobially equipped implant which avoids disadvantages known from the state of the art. In particular, the implant provided by the invention should afford long-term antimicrobial protection without mechanical properties of the implant being adversely affected thereby.

This problem is solved according to the invention by a medical implant with the features of the independent claim 1. Preferred embodiments of the implant are the subject of the dependent claims 2 to 22. The present invention further relates also to production processes for the implant as claimed in the independent claims 23 and 25. A preferred embodiment for a production process is the subject of the dependent claim 24. A further aspect of the invention relates to the use according to independent claim 26. The wording of all claims is by reference hereby made part of the content of this description.

The implant according to the invention is a medical implant equipped with an antimicrobial composition which comprises silicon dioxide and metal-containing nanoparticles. According to the invention, it can in particular be provided that the composition is an antimicrobial composition made of silicon dioxide and metal-containing nanoparticles.

Through the invention, an antimicrobially equipped medical implant is provided whose mechanical properties, in particular whose strength or mechanical resistance, remain essentially unaffected by the anti-microbial equipping. Further, the implant especially advantageously provides a uniform and in particular long-persisting antimicrobial protection, owing to which the implant is also a powerful alternative to implants known from the state of the art with regard to the avoidance of late infections. Particularly advantageously, the antimicrobial composition used in the context of the invention is a biocompatible composition so that bioavailability of the implant according to the invention is also guaranteed.

In principle, the antimicrobial equipping of the implant can mean a coating or impregnation of the implant with the composition. In a possible embodiment, therefore, the implant is coated or impregnated with the antimicrobial composition, in particular on the implant surface. The coating or impregnation can in addition contain binders which improve the adhesion to the implant.

The antimicrobial composition is preferably incorporated into a material, in particular into a polymeric material, of the implant. The antimicrobial composition can be present finely divided in the implant. The antimicrobial composition is preferably present dispersed, in particular homogeneously dispersed, in the implant. According to the invention it can also be provided that the composition is finely divided in the interior of the implant and preferably also on its surface. Through the fine, in particular dispersed, distribution of the composition in the implant, a uniform and in particular controlled release of the antimicrobial composition, in particular of the metal-containing nanoparticles, into the surrounding tissue after implantation is possible with particular advantage. The metal-containing nanoparticles can be released in ionogenic or non-ionogenic form. As a rule, the metal-containing nanoparticles are released in ionogenic form. The ionogenic form is usually created by contact with water or body fluids. Here, nano-particles present in ionogenic form exhibit an anti-microbial, in particular antibacterial, action on their surface. Particularly advantageously, the fine, in particular dispersed, distribution of the composition is also maintained with high contents of composition in the implant.

In a further embodiment, the metal-containing nanoparticles are present dispersed in the implant. The nanoparticles can in particular be present dispersed in the silicon dioxide. The nanoparticles can at least be present dispersed in the antimicrobial composition. The silicon dioxide itself is preferably amorphous, in particular X-ray amorphous. The silicon dioxide is preferably present in the composition in an amorphous form.

The antimicrobial composition itself is usually present in the form of a powder, in particular in the form of a fine, light and in particular dusty powder. Depending on the metal-containing nanoparticles, the antimicrobial composition can have a coloration, or at least a color cast. For example, in the case of silver nanoparticles, the composition is light yellowish or brownish colored. Any coloration of the antimicrobial composition is mainly dependent on the content of the nanoparticles in the composition. According to the invention, it can also be provided that the implant has a coloration caused by the antimicrobial composition.

In a further embodiment, the metal-containing nanoparticles are present in the implant, in particular also in the silicon dioxide, in the form of individual, in other words discrete, particles. In this embodiment the implant is especially advantageously characterized in that it is essentially free of agglomerates or aggregates of the nanoparticles. As a result, an adverse effect on the mechanical stability of the implant can be avoided.

In a preferred embodiment, the silicon dioxide has a three-dimensional basic structure, in particular of the matrix type, in the antimicrobial composition. The basic structure is typically formed of particles of the silicon dioxide. The basic structure advantageously has cavities which can be linked to one another by channels. This basic structure, i.e. its surface and/or cavities or channels thereof, preferably contains the metal-containing nanoparticles.

The silicon dioxide typically comprises particles with a diameter between 1 and 50 nm, in particular 5 and 30 nm, preferably 10 and 20 nm. According to the invention, it can further be possible that the silicon dioxide, in particular in the antimicrobial composition, comprises agglomerates. Preferably the silicon dioxide comprises agglomerates (aggregates) of silicon dioxide particles. Particularly preferably, the silicon dioxide comprises mixed agglomerates, in particular based on silicon dioxide particles and metal-containing nanoparticles. The agglomerates can be organized into higher order chain-like, in particular necklace-like, structures. The chains or chainlike structures can be further assembled into a higher order structure with cavities and linking channels. The agglomerates preferably have a size which essentially does not influence the mechanical properties of the implant, in particular its mechanical stability or strength. As a rule, the agglomerates have a diameter between 50 and 2000 nm, in particular 100 and 1000 nm. In addition, the agglomerates are usually rather loose composite structures which can be readily driven apart again for example under mechanical stress. As a result, the silicon dioxide and hence preferably also the metal-containing nanoparticles can be particularly well dispersed in a material which is used for producing the implant.

In a suitable embodiment, the antimicrobial composition, in particular mixed agglomerates of silicon dioxide particles and the metal-containing nanoparticles, has a high specific surface area, in particular a specific surface area between 100 and 400 m²/g.

The nanoparticles preferably have a diameter between 5 and 20 nm. However, embodiments wherein the nanoparticles have a diameter<5 nm and/or >20 nm are possible.

The content of the metal-containing nano-particles in the antimicrobial composition is preferably between 2 and 40 wt. %, in particular 5 and 20 wt. %, based on the total weight of the composition. The content of silicon dioxide in the antimicrobial composition is preferably between 98 and 60 wt. %, in particular 95 and 80 wt. %, based on the total weight of the composition.

In a further embodiment, the content of the metal-containing nanoparticles in the implant is between 10 and 5000 ppm, based on the total weight of the implant. Higher contents of metal-containing nanoparticles as a rule result in no appreciable improvement in the antimicrobial protection of the implant. Nonetheless, higher nanoparticle contents can result in increased washing resistance and hence in general in longer antimicrobial protection. Moreover, higher contents of the metal-containing nanoparticles can be preferred in implants which have a low surface to volume ratio. The content of the metal-containing nanoparticles is preferably between 50 and 2000 ppm, especially preferably 200 and 1500 ppm, based on the total weight of the implant.

The content of silicon dioxide in the implant usually corresponds to about 4 to 20 times the nano-particle content in the implant. Preferably, the content of silicon dioxide in the implant is between 40 and 100 000 ppm, in particular 1000 and 10 000 ppm, preferably 3000 and 6000 ppm, particularly preferably 4000 and 5000 ppm, based on the total weight of the implant.

As a rule the metal-containing nanoparticles have antimicrobial activity or antimicrobial properties. The metal-containing nanoparticles are preferably formed of antimicrobially active metals and/or metal salts, in particular metal oxides. Thus the nanoparticles can in particular be formed of gold, silver, copper, zinc, titanium and/or salts, preferably oxides, of these metals. With regard to titanium dioxide as a possible metal oxide, it should be stated that this metal oxide as a rule only displays an anti-microbial action in the presence of oxygen and light. Particularly preferably, the nanoparticles are formed of silver, copper, silver oxide and/or copper oxide.

As already mentioned, the implant according to the invention particularly advantageously affords long-term antimicrobial protection. The implant preferably affords antimicrobial protection over a period of several months up to several years. In the case of so-called permanent implants, antimicrobial protection preferably exists at least over a period of 10 to 15 years.

Concerning further features and details about the silicon dioxide and the metal-containing nanoparticles, reference is made to the publications WO 2006/084411 A1, WO 2006/084390 A1 and EP 1 889 810 A1, the disclosure content whereof is by express reference made part of the content of this description.

In a further embodiment, the antimicrobial composition is producible and/or produced by a flame spray pyrolysis process. For this, in a first step a solution of a metal salt and a preferably readily volatile silicon compound in an organic solvent is prepared. As suitable silicon compounds, in particular organic silanes, for example tetraethoxyorthosilane and/or hexamethyldisiloxane are possible. Suitable solvents are alcohols, in particular methanol, ethanol, n-propanol, n-butanol, isopropanol, ethanediol, propanediol and mixtures thereof. If necessary an ultrasound treatment can be applied in order to promote the dissolution of the metal salt in the solvent. In a second step, the solution is sprayed into a flame with a temperature of ca. 1500° C. The flame is usually ignited by means of a gas mixture, for example of methane and oxygen. Thereafter the flame sustains itself by combustion of the solution. The deposition of the antimicrobial composition, in particular in the form of fine particles, preferably takes place on water-cooled filter systems, on which moreover a reduced pressure can be applied. Concerning further features and details about the flame spray pyrolysis process, reference is made to the publications WO 2006/084411 A1, WO 2006/084390 A1 and EP 1 889 810 A1, the respective disclosure content whereof in this respect is by express reference made part of the content of this description.

The implant is preferably formed of polymeric materials. The materials can in general be homo-, co-, tri- or tetrapolymers, or the like. The materials can in particular be present as block copolymers or block terpolymers. The term material in the sense of the present invention should be understood to mean the natural material (raw material without the anti-microbial composition).

The implant can in principle be formed of an absorbable or non-absorbable material. The implant is preferably formed of a non-absorbable material. Non-absorbable materials are particularly advantageous when long-term antimicrobial protection and in particular a low dosage release of antimicrobially active components of the composition, as a rule the metal-containing nanoparticles, should be attained. As non-absorbable materials, the materials normally used for the production of non-absorbable implants, preferably polymers, are possible. As non-absorbable materials, polyolefins, polyesters and also in many cases polyamides are above all possible. The non-absorbable materials are preferably polyethylene, polypropylene, polytetrafluoroethylene, polyethylene terephthalate and/or polyurethane. The polytetrafluoroethylene is preferably expanded polytetrafluoroethylene.

Alternatively to or in combination with the materials described above, in a possible embodiment the implant is formed of an absorbable material. As suitable absorbable materials, polymers, in particular co- or terpolymers, preferably block copolymers or block terpolymers, based on hydroxycarboxylic acid units are above all possible. Polymers based on lactide, glycolide, trimethylene carbonate, ε-capro-lactone, paradioxanone and/or hydroxybutyric acid are preferred.

In a further embodiment, the implant is formed partly of a non-absorbable material and partly of an absorbable material. Concerning the possible materials, reference is made in particular to the above embodiments.

According to the invention, it can further be provided that as well as the antimicrobial composition the implant contains further additives. The additives can for example be binders, antibiotics, inflammation-inhibiting active substances, odor-suppressing active substances, disinfectants, growth factors or the like. According to the invention, it can also be provided that the implant contains an X-ray contrast agent, in particular a barium-containing compound, for example barium sulfate.

In a preferred embodiment, the implant is a textile implant, in particular a textile flat shape. The implant can for example be a woven fabric, hosiery, knitted fabric, nonwoven or fleece.

According to a further embodiment, the implant contains fibers, in particular polymer fibers, with a titer between 3 and 3500 dtex, in particular 20 and 1000 dtex, preferably 50 and 250 dtex. The fibers are typically mono- and/or multifil fibers. In principle, the fibers can have a diameter between 10 μm and 1.3 mm. The diameters preferably lie in the range between 0.04 and 0.90 mm. As fiber gauges, the fiber gauges normally used, in particular USP-8/0, USP-7/0, USP-6/0, USP-5/0, USP-4/0, USP-3/0, USP-2/0, USP-0, USP-1, USP-2, USP-3, USP-4, USP-5 and/or USP-6, preferably USP-8/0, USP-7/0, USP-6/0, USP-5/0, USP-4/0, USP-3/0, USP-2/0, USP-0, USP-1 and/or USP-2 are possible.

The implant can further contain fibers which for example have a strength between 30 and 90 cN/tex, in particular 40 and 80 cN/tex, preferably 50 and 70 cN/tex. Here the term strength should be understood to mean the breaking strain of the fibers.

Further, it can be preferred that the implant is porous, and in particular has pores with a pore size between 0.05 and 2 mm. The implant can also have an interconnecting porosity.

In a particularly preferred embodiment, the implant is a surgical suture material. The suture material can be mono- or multifil. In addition, it can according to the invention be provided that the implant is a braided surgical suture material. Concerning further properties and features of the suture material, in particular with regard to fibers, fiber gauges and titers, reference is made to the above description.

According to a further preferred embodiment, the implant is present in the form of a replacement hollow organ, in particular in the form of a vascular prosthesis. Further, it can be preferred that the implant is a textile net, in particular hernia net, prolapse net or urinary incontinence net. The implant can further be present as a band, for example as a urinary incontinence band.

In a further possible embodiment, the implant is present as a film, sponge, foam, membrane, access port, catheter, hose, syringe, cannula or hemostatic means. The cannula can be an indwelling cannula, in particular a venous indwelling cannula. The hemostatic means can for example be formed as a plaster, bandage or wound dressing.

The invention further relates to a process for producing the implant according to the invention, wherein an antimicrobial composition, in particular in the form of a master batch or compound, comprising silicon dioxide and metal-containing nanoparticles, is added into a material of the implant during the production thereof. In other words, during the process the antimicrobial composition provided according to the invention is incorporated into an implant material with the formation of the implant according to the invention.

The master batch usable in the context of the present invention is preferably a concentrate of the antimicrobial composition in a polymeric material which can be present in the form of granules or pellets. The polymeric material of the master batch is preferably the material which is used for producing the implant. The granules or pellets usually have a higher content of the composition than the implant produced.

For the production of the master batch, the antimicrobial composition is mixed with a polymeric material. The mixture is usually melted in an extruder and then extruded. The final shaping of the master batch is effected by granulation of the extruded material. The extruded material can be further processed into granules or pellets for example by strand granulation or underwater granulation.

In the context of the invention, a compound should be understood to mean a polymeric material of the implant which already contains the desired quantity of antimicrobial composition. As a rule the compound is further processed to the implant according to the invention by suitable processes.

If necessary during the production of a master batch or compound, dispersant additives can be used in order to enable or aid better mixing, in particular dispersion, of the antimicrobial composition in a polymeric material. Now the techniques for producing master batches and compounds are sufficiently well known to the person skilled in the art, so that a detailed description will not be given here.

In a preferred embodiment, the antimicrobial composition is mixed with the material, in particular a polymer, and then shaped into an implant according to the invention, in particular extruded, spun, pressed, rolled, cast or blown. Preferably, a mixture of the implant material and the antimicrobial composition, which is advantageously present in the form of a master batch, is spun out in the form of a fiber material. As suitable spinning techniques for this, all methods familiar to the person skilled in the art are possible, in particular melt, gel, solution and in some cases dry spinning. The spinning preferably takes place from the melt which contains the material of the implant and the antimicrobial composition. The spun fiber material can be further processed either into a surgical suture material or another textile implant. Concerning this, reference is made to the above description.

In addition, the invention relates to a process for producing the implant according to the invention, wherein an antimicrobial composition comprising silicon dioxide and metal-containing nanoparticles is applied from outside onto a non-antimicrobially equipped implant, in particular onto the surface thereof. The implant is preferably coated with the composition. For application of the composition, the non-antimicrobially equipped implant can for example be dipped into a dipping bath which contains the composition. Alternatively, or in combination with this, the composition can also be sprayed onto the implant.

The implant produced is particularly suitable for use in surgery and therein in particular for the surgical care of wounds.

Finally, the invention also relates to the use of an antimicrobial composition comprising silicon dioxide and metal-containing nanoparticles for producing an implant according to the invention. Concerning further features and details, in particular with regard to the composition, the silicon dioxide and/or the metal-containing nanoparticles, reference is made to the above description.

Further features of the invention emerge from the following description of preferred embodiments on the basis of figures and examples. Here the individual features of the invention can be implemented alone or in combination with one another. The embodiments described serve for explanation and better under-standing of the invention and are in no way to be understood as limiting.

In the figures:

FIG. 1: shows the antimicrobial properties of a polypropylene fiber according to the invention with various silver loadings against various microorganisms,

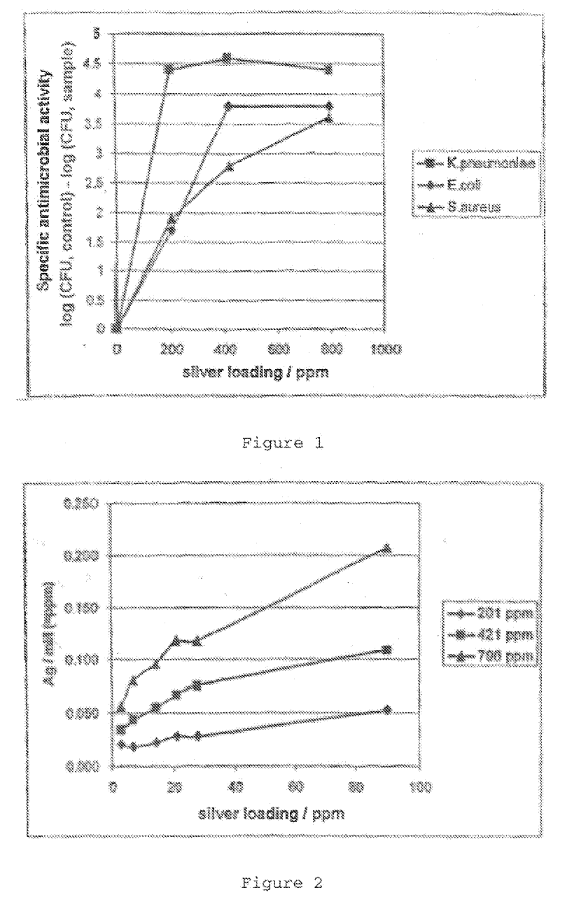

FIG. 2: shows the release kinetics of silver from nets according to the invention,

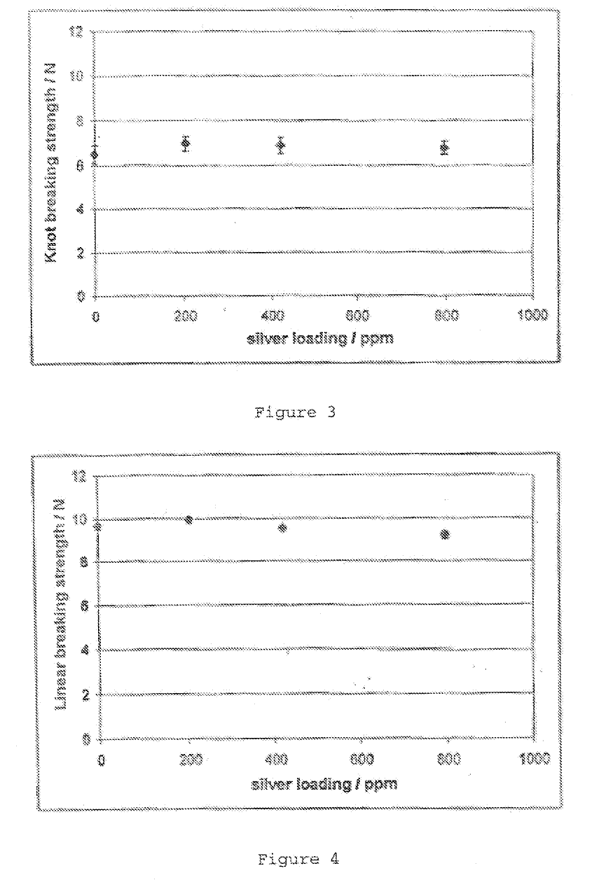

FIG. 3: shows the effect of various silver loadings on the knot breaking strength of a polypropylene fiber according to the invention,

FIG. 4: shows the effect of various silver loadings on the linear breaking strength of a poly-propylene fiber according to the invention,

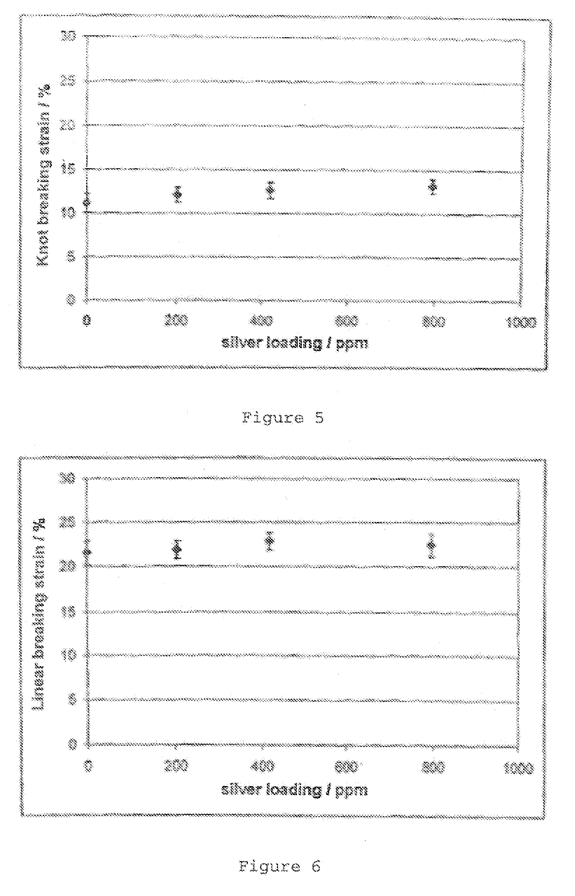

FIG. 5: shows the effect of various silver loadings on the knot breaking strain of a poly-propylene fiber according to the invention,

FIG. 6: shows the effect of various silver loadings on the linear breaking strain of a poly-propylene fiber according to the invention.

### EXAMPLE 1

### Production of a Master batch from Silver, Silicon Dioxide and Polypropylene

10 kg of medical grade polypropylene were placed beforehand in a double-screw extruder and melted at a temperature of ca. 180° C. 250 g of a composition of amorphous silicon dioxide and silver nanoparticles containing 20 wt. % silver was then mixed into the molten polypropylene, so that the content of the composition in the mixture was ca. 2.43 wt. %. Next, the molten mixture was extruded to produce granules. This material had a silver content of ca. 4800 ppm.

### EXAMPLE 2

### Antimicrobial Equipping of a Surgical Suture Material

A total of three antimicrobially equipped suture materials were produced. For this, three extrusions were performed, wherein a master batch appropriately prepared according to example 1 with a silver content of ca. 4600 ppm was added in different quantities to a polypropylene melt before the extrusion.

In the first batch, a quantity of master batch was added such that the mixture had a master batch content of ca. 4.3 wt. %, based on the total weight of the raw polymer used (pure polypropylene). The spun fiber material had a silver content of 201 ppm, based on the total weight of the fiber material.

In the second batch, a quantity of master batch was added such that the mixture had a master batch content of ca. 10.9 wt. %, based on the total weight of the raw polymer used (pure polypropylene). The spun fiber material had a silver content of 421 ppm, based on the total weight of the fiber material.

In the third batch, a quantity of master batch was added such that the mixture had a master batch content of ca. 21.7 wt. %, based on the total weight of the raw polymer used (pure polypropylene). The spun fiber material had a silver content of 796 ppm, based on the total weight of the fiber material.

### EXAMPLE 3

### Antimicrobial Tests

A suture material with a silver content of 2600 ppm, based on the total weight of the suture material, produced from polypropylene according to example 2 was subjected to an antimicrobiological test according to the Japanese industrial standard for the antibacterial activity and effectiveness on textile products (JIS L 1902). For this, ca. 0.4 g of a sample of the suture material was wound into tight filament bundles. The bundles were sterilized with 70% ethanol and then dried. Next, each filament bundle was inoculated with ca. 50 μl of a bacterial culture which contained ca. 4×10⁶ colony forming units per cm³ of Klebsiella pneumoniae (DSM 789), and incubated at ca. 37° C. for 18 hrs. Next, the bacteria still living were rinsed from each filament bundle and counted. The results thus obtained are shown in table 1 in table form.

TABLE 1

| | | | Decrease in |
|---|---|---|---|
| | Time | CFU (colony | bacterial |
| | (hrs) | forming units) | colonization [%] |
| Bacteria-inoculated samples | 0 | 4.1 × 10⁶ | — |
| without Ag—SiO₂ | | | |
| composition | | | |
| Untreated control sample | 18 | 4.9 × 10⁵ | — |
| Bacteria-inoculated samples | 18 | <9.9 × 10¹ | >99.99 |
| with the Ag—SiO₂ | | | |
| composition | | | |

Further antimicrobial tests with surgical suture materials treated according to the invention are shown in table 2 in table form:

TABLE 2

| | | | CFU | Decrease in |
|---|---|---|---|---|
| Polymer/ | | | (colony | bacterial |
| Ag | | Time | forming | coloniza- |
| (ppm) | | (hrs) | units) | tion [%] |
| Poly- | Bacteria-inoculated | 0 | 1.5 × 10⁶ | — |
| styrene/ | samples without | | | |
| 500 | Ag—SiO² composition | | | |
| | Untreated control | after | 2.6 × 10⁷ | — |
| | sample | 24 | | |
| | | hrs | | |
| | Bacteria-inoculated | after | <9.9 × 10¹ | >99.99 |
| | samples with the | 24 | | |
| | Ag—SiO₂ composition | hrs | | |
| Polypro- | Bacteria-inoculated | 0 | 2.3 × 10⁵ | |
| pylene/ | samples without | | | |
| 1000 | Ag—SiO₂ composition | | | |
| | Untreated control | after | 6.7 × 10⁶ | — |
| | sample | 24 | | |
| | | hrs | | |
| | Bacteria-inoculated | after | <9.9 × 10¹ | >99.99 |
| | samples with the | 24 | | |
| | Ag—SiO₂ composition | hrs | | |
| Poly- | Bacteria-inoculated | 0 | 1.5 × 10⁶ | |
| amide | samples without | | | |
| 6/500 | Ag—SiO₂ composition | | | |
| | Untreated control | after | 1.1 × 10⁷ | — |
| | sample | 24 | | |
| | | hrs | | |
| | Bacteria-inoculated | after | <9.9 × 10¹ | >99.99 |
| | samples with the | 24 | | |
| | Ag—SiO₂ composition | hrs | | |

### EXAMPLE 4

### Cytotoxicity Test

The polypropylene fibers produced in example 2 were subjected to a test for cytotoxicity according to ISO 10993. In this, the cytotoxic action was assessed on the basis of the following grading:

Grade 0: no cytotoxicity, grade 1: insignificant cytotoxicity, grade 2: weak cytotoxicity, grade 3: moderate cytotoxicity and grade 4: severe cytotoxicity.

In table 3 below, the results obtained in the test are shown in table form.

TABLE 3

| Silver loading/ppm | Cytotoxicity grade |
|---|---|
| 0 | 0 |
| 201 | 0 |
| 421 | 0 |
| 796 | 0 |

The results confirm the biocompatible properties of the antimicrobial composition used in the context of the invention.

### DESCRIPTION OF DIAGRAMS

FIG. 1 is a graph showing the antimicrobial activity of a ca. 150 μm thick monofil polypropylene fiber which is antimicrobially treated according to the present invention, against various microorganisms. Here the antimicrobial activity is shown on the y axis as the difference between the logarithm to the base ten of the colony forming units (CFU) of a reference fiber and the logarithm to the base ten of the colony forming units (CFU) of the antimicrobially equipped fiber, and the silver loading (ppm) is shown on the x axis. FIG. 1 already shows a marked antimicrobial activity at silver loadings of the fiber of markedly <1000 ppm. This applies above all with reference to the micro-organisms Klebsiella pneumoniae and Escherichia coli, where silver loadings of ca. 400 ppm already lead to high antimicrobial activity of the fiber. In the case of Staphylococcus aureus and Escherichia coli a kill of >99.9% could be observed with a silver loading of the fiber of ca. 800 ppm. In the case of Klebsiella pneumoniae, also at a silver loading of the fiber of ca. 800 ppm, a kill of >99.99% was observed. The study was based on the test method JIS L 1902 or DIN EN ISO 20743.

FIG. 2 is a graph showing the measured release kinetics of silver from textile nets which were produced from fibers as described in example 2. For the determination of the release kinetics, net pieces (ca. 1.3 g) were extracted in 40 ml of a 0.9% sodium chloride solution at 37° C. and the silver content of the solution was then determined. The values shown in the graph in FIG. 2 substantiate a continuous release of silver over at least 90 days.

FIG. 3 is a graph showing the effect of the antimicrobial composition described in more detail in example 1 on the knot breaking strength of a monofil polypropylene fiber with a diameter of ca. 150 μm. For this the knot breaking strength (measured in Newtons, plotted on the y axis of FIG. 3) was measured as a function of the silver loading (measured in ppm, plotted on the x axis of FIG. 3). Concerning this, FIG. 3 shows that the antimicrobial composition results in no significant change in the knot breaking strength of the polypropylene fiber.

FIG. 4 is a graph showing the effect of the antimicrobial composition described in more detail in example 1 on the linear breaking strength of a monofil polypropylene fiber with a diameter of ca. 150 μm. For this the linear breaking strength (measured in Newtons, plotted on the y axis of FIG. 4) was measured as a function of the silver loading (measured in ppm, plotted on the x axis of FIG. 4). The data shown in the graph in FIG. 4 show clearly that the antimicrobial composition provided according to the invention results in no appreciable impairment of the linear breaking strength of the polypropylene fiber.

FIG. 5 is a graph showing the effect of the antimicrobial composition described in more detail in example 1 on the knot breaking strain of a monofil polypropylene fiber with a diameter of ca. 150 μm. For this the knot breaking strain (measured in percent, plotted on the y axis of FIG. 5) was measured as a function of the silver loading (measured in ppm, plotted on the x axis of FIG. 5). The data shown in the graph in FIG. 5 show that the silver loading of the fiber also results in no appreciable impairment of its mechanical properties in this respect in comparison to a silver-free fiber equipped with the composition.

FIG. 6 is a graph showing the effect of the antimicrobial composition described in more detail in example 1 on the linear breaking strain of a monofil polypropylene fiber with a diameter of ca. 150 μm. For this the linear breaking strain (measured in percent, plotted on the y axis of FIG. 6) was measured as a function of the silver loading (measured in ppm, plotted on the x axis of FIG. 6). Here the breaking strain is a measure of the relative proportion to which the fiber was elongated at break. The data reproduced in the graph in FIG. 6 show that the antimicrobial composition provided according to the invention also results in no appreciable impairment of the linear breaking strain.

Summarizing, it can thus be stated that the antimicrobial composition usable in the context of the present invention results in no appreciable impairment of the mechanical properties of the fiber.

1. A medical implant comprising an antimicrobial composition which comprises silicon dioxide and metal-containing nanoparticles.

2. The medical implant of claim 1, wherein the antimicrobial composition is dispersed in the implant.

3. The medical implant of claim 1, wherein the metal-containing nanoparticles are dispersed in the silicon dioxide.

4. The medical implant of claim 1, wherein the silicon dioxide is X ray amorphous.

5. The medical implant of claim 1, wherein the silicon dioxide has a three-dimensional matrix basic structure.

6. The medical implant of claim 1, wherein the silicon dioxide is present in the form of particles with a diameter between 1 and 50 nm.

7. The medical implant of claim 1, wherein particles of the silicon dioxide together with the metal-containing nanoparticles are present in the form of agglomerates.

8. The medical implant of claim 1, wherein the anti-microbial composition has a specific surface area between 100 and 400 m²/g.

9. The medical implant of claim 1, wherein the metal-containing nanoparticles have a diameter between 5 nm and 20 nm.

10. The medical implant of claim 1, wherein the content of the metal-containing nanoparticles in the antimicrobial composition is between 2 and 40 wt. % based on the total weight of the composition.

11. The medical implant of claim 1, wherein the content of the silicon dioxide is between 98 and 60 wt. % based on the total weight of the composition.

12. The medical implant of claim 1, wherein the content of the metal-containing nanoparticles is between 10 and 5000 ppm based on the total weight of the implant.

13. The medical implant of claim 1, wherein the content of the silicon dioxide is between 40 ppm and 100 000 ppm, based on the total weight of the implant.

14. The medical implant of claim 1, wherein the metal-containing nanoparticles are formed of gold, silver, copper, zinc, titanium and/or salts thereof.

15. The medical implant of claim 1, wherein the composition is preparable or prepared by a flame spray pyrolysis process.

16. The medical implant of claim 1, wherein the implant is a textile flat shape product.

17. The medical implant of claim 1, wherein the implant is a woven fabric, hosiery, knitted fabric, nonwoven or fleece.

18. The medical implant of claim 1, further comprising fibers with a titer between 3 and 3500 dtex.

19. The medical implant of claim 1, further comprising fibers with a strength between 30 and 90 cN/tex.

20. The medical implant of claim 1, wherein the implant is a surgical suture material.

21. The medical implant of claim 1, wherein the implant is a textile hernia net, prolapse net or a net for urinary incontinence.

22. The medical implant claim 1, wherein the implant is a vascular prosthesis.

23. A process for producing the medical implant of claim 1, comprising adding an antimicrobial composition in the form of a master batch or compound comprising silicon dioxide and metal-containing nanoparticles to a material of the implant during the production thereof.

24. The process as claimed in claim 23, wherein the antimicrobial composition is mixed with the material and shaped, extruded, spun, pressed, rolled, cast or blown.

25. A process for producing the medical implant of claim 1, comprising applying an antimicrobial composition comprising silicon dioxide and metal-containing nanoparticles from outside onto a non-antimicrobially equipped implant.

26. (canceled)
